(19) United States
(12) Patent Application Publication
(10) Pub. No.: US 2026/0240747 A1
(43) Pub. Date: Aug. 20, 2026

(54) COSMETIC MATERIAL AND BEAUTY CARE METHOD

(71) Applicant: SHISEIDO COMPANY, LTD., Chuo-ku, Tokyo (JP)

(72) Inventor: Mika YOSHIMURA, Tokyo (JP)

(73) Assignee: SHISEIDO COMPANY, LTD., Chuo-ku, Tokyo (JP)

(21) Appl. No.: 18/992,679

(22) PCT Filed: Feb. 16, 2023
(86) PCT No.: PCT/JP2023/005463

§ 371 Date: Jan. 9, 2025

(30) Foreign Application Priority Data
Aug. 9, 2022 (JP) 2022-127207

(51) Int. Cl. A61K 8/73; A61K 8/19; A61Q 5/12; A61Q 19/00

(52) U.S. Cl. CPC A61K 8/735; A61K 8/19; A61Q 5/12; A61Q 19/007; A61K 2800/80

(57) ABSTRACT

A subject matter of the present disclosure is to provide a cosmetic that can exhibit better skin penetrability and a better moisture-retaining property using a compacted hyaluronic acid, and a beauty method using the cosmetic.

A cosmetic of the present disclosure comprises a hyaluronic acid and a magnesium salt, wherein the magnesium salt is contained at a concentration corresponding to an ionic strength of 0.01 or more, and wherein the mass ratio of the magnesium salt to the hyaluronic acid is 1.0 or more.

### FIELD

The present disclosure relates to a cosmetic and a beauty method.

### BACKGROUND

For the purpose of, for example, moisturization of the skin, hyaluronic acid, which has a moisturizing function, has been utilized in the fields of cosmetics and the like.

PTL 1 discloses a cosmetic comprising a hyaluronic acid-loaded nanoparticle in which hyaluronic acid is loaded in/on at least one of the insides or the surface of a nanoparticle formed of polylactic acid, polyglycolic acid, or a lactic acid-glycolic acid copolymer.

PTL 2 discloses a skin external preparation, comprising a composite nanoparticle containing: (A) hyaluronic acid; and (B) a zwitterionic compound, wherein the particle diameter is 100 nm or less.

PTL 3 discloses a cosmetic comprising: an aqueous medium; and hyaluronic acid particles dispersed in the aqueous medium, wherein the average particle diameter of the hyaluronic acid particles is 200 nm or less.

### CITATION LIST

### Patent Literature

[PTL 1] Japanese Unexamined Patent Publication (Kokai) No. 2010-150151
[PTL 2] WO 2018/182003
[PTL 3] WO 2021/033725

### SUMMARY

### Technical Problem

Hyaluronic acid has a negative charge due to the presence of carboxyl groups. Due to the electrostatic repulsion based on this negative charge, hyaluronic acid is generally in the form of spread threads.

Compacted hyaluronic acids, such as those described in PTLs 1 and 2, are more likely to stay in grooves, pores, and the like of the skin compared to hyaluronic acids in the form of spread threads. However, since the content ratio of hyaluronic acid in the particles described in PTL 1 is as low as about 3% by mass, and the content ratio of hyaluronic acid in the particles described in PTL 2 is only 50% by mass or less, there have been cases where a sufficient moisture-retaining property could not be secured.

As described in PTL 3, researchers of the present applicant discovered that the electrostatic shielding effect of sodium chloride can be utilized to apparently neutralize the carboxyl-derived negative charge of hyaluronic acid, to achieve dominance of hydrogen bonds, which have an attracting action, based on hydroxyl groups and the like in the hyaluronic acid, to thereby suppress spreading of the hyaluronic acid and enable compaction of the hyaluronic acid.

In Patent Document 3, compaction of hyaluronic acid was substantially achieved using sodium chloride. However, a compaction technique for hyaluronic acid that can achieve an even higher moisture-retaining property than that of the hyaluronic acid prepared in such a manner has been demanded.

In view of this, a main object of the present disclosure is to provide a cosmetic that can exhibit better skin penetrability and a better moisture-retaining property using a compacted hyaluronic acid, and a beauty method using the cosmetic.

### Solution to Problem

### <Aspect 1>

A cosmetic comprising:
a hyaluronic acid; and
a magnesium salt,
wherein the magnesium salt is contained at a concentration corresponding to an ionic strength of 0.01 or more, and
wherein the mass ratio of the magnesium salt to the hyaluronic acid is 1.0 or more.

### <Aspect 2>

The cosmetic according to aspect 1, wherein the hyaluronic acid is contained at 0.005% by mass or more with respect to the total amount of the cosmetic.

### <Aspect 3>

The cosmetic according to aspect 1 or 2, wherein the magnesium salt is magnesium chloride.

### <Aspect 4>

The cosmetic according to any one of aspects 1 to 3, wherein the weight average molecular weight of the hyaluronic acid is 10,000,000 or less.

### <Aspect 5>

The cosmetic according to any one of aspects 1 to 4, which is a leave-on type cosmetic.

### <Aspect 6>

The cosmetic according to any one of aspects 1 to 5, having an L value of 90 or more.

### <Aspect 7>

The cosmetic according to any one of aspects 1 to 6, for use in penetration of hyaluronic acid into body surface or body hair.

### <Aspect 8>

A beauty method comprising applying the cosmetic according to any one of aspects 1 to 7 to body surface or body hair.

### <Aspect 9>

The beauty method according to aspect 8, wherein the application of the cosmetic to the body surface or body hair is carried out for two or more days.

### <Aspect 10>

The beauty method according to aspect 8, wherein the application of the cosmetic to the body surface or body hair is carried out continuously for two or more days.

### Advantageous Effects of Invention

According to the present disclosure, a cosmetic that can exhibit better skin penetrability and a better moisture-retaining property using a compacted hyaluronic acid, and a beauty method using the cosmetic, can be provided.

### BRIEF DESCRIPTION OF DRAWINGS

FIG. 1 is a graph related to the amount of hyaluronic acid in human skin as observed when aqueous hyaluronic acid solutions prepared using various salts were applied to the human skin.

FIG. 2 is (a) a photograph obtained by applying an aqueous hyaluronic acid solution containing no magnesium salt to human skin, and observing a cross-section of the skin under a fluorescence microscope, and (b) a photograph obtained by applying an aqueous solution containing a hyaluronic acid compacted using a magnesium salt to human skin, and observing a cross-section of the skin under a fluorescence microscope.

FIG. 3 is a graph related to the amount of hyaluronic acid in human skin to which hyaluronic acid was not applied, and the amount of hyaluronic acid in human skin as observed when an aqueous hyaluronic acid solution or an aqueous solution containing hyaluronic acid and a magnesium salt was applied to the human skin continuously for one month.

### DESCRIPTION OF EMBODIMENTS

Hereinafter, embodiments of the present disclosure are described in detail. The present disclosure is not limited to the following embodiments, and may be variously modified and practiced within the scope of the present invention.

The cosmetic of the present disclosure comprises hyaluronic acid and a magnesium salt, wherein the magnesium salt is contained at a concentration corresponding to an ionic strength of 0.01 or more, and wherein the mass ratio of the magnesium salt to the hyaluronic acid is 1.0 or more.

The action principle by which the inclusion of the hyaluronic acid and the magnesium salt at the prescribed ratios enables the compaction of the hyaluronic acid, and by which the better skin penetrability and the better moisture-retaining property can be achieved compared to cases where other salts such as a sodium salt are used, is thought to be as follows, although the present invention is not limited by the principle. Here, “compaction” in the present disclosure means that hyaluronic acid is shrunk compared to one molecule of hyaluronic acid, or compared to a structure in a state where hyaluronic acid molecules are tangled together, the molecule or structure being in a state without addition of a magnesium salt. Typically, one molecule of hyaluronic acid, or a structure in a state where hyaluronic acid molecules are tangled together, can be said to be shrunk in cases where a magnesium salt is contained in a prescribed amount together with the hyaluronic acid.

Hyaluronic acid generally has a negative charge due to the presence of carboxyl groups. Due to the electrostatic repulsion based on this negative charge, the molecule of hyaluronic acid was likely to be, for example, in the form of a spread thread, so that the molecule tended not to be compacted. Therefore, compaction of a structure of a hyaluronic acid molecule(s) by shrinkage required, as in the cases of PTLs 1 and 2, a nanoscale support material capable of supporting the hyaluronic acid molecule(s). As a result, the content ratio of hyaluronic acid in the compacted structure has been limited.

In contrast, according to the technique of PTL 3, the electrostatic shielding effect by sodium chloride can be utilized to apparently neutralize the negative charge of hyaluronic acid. As a result, dominance of hydrogen bonds can be achieved to suppress spreading of the hyaluronic acid molecule(s), so that compaction of even a hyaluronic acid molecule(s) alone is possible. Since the compacted structure of hyaluronic acid prepared by the technique of PTL 3 can be obtained without using the support material described above, the content ratio of hyaluronic acid in the structure can be theoretically 100% by mass.

Since the compacted hyaluronic acid prepared by the technique of PTL 3 has an average particle diameter of 200 nm or less, the hyaluronic acid is capable of penetrating into the skin more easily than non-compacted hyaluronic acid, so that improved skin penetrability and an improved moisture-retaining property can be achieved. However, the present inventor discovered that, in cases where a magnesium salt, among a wide variety of salts, is used as the salt for the compaction of hyaluronic acid, better skin penetrability and a better moisture-retaining property can be achieved compared to PTL 3, which used sodium chloride.

Although the reason is unclear, one possible factor that affects the above fact may be a difference in the crystallinity of the salt used. More specifically, among salts, sodium salt is a salt whose crystallization relatively easily occurs. In contrast, magnesium salt is a salt whose crystallization is less likely to occur compared to sodium salt since magnesium salt is deliquescent. Therefore, in the case of a cosmetic containing hyaluronic acid, a sodium salt, and water, application of the cosmetic is thought to cause formation of a crystallized film on the skin surface by the sodium salt as the moisture in the applied cosmetic decreases due to drying. As a result, at least part of the compacted hyaluronic acid may be immobilized in this crystallized film, and hence may be incapable of penetration into the skin. On the other hand, in the case of a cosmetic containing hyaluronic acid, a magnesium salt, and water, even when the cosmetic is applied to the skin followed by evaporation of the moisture in the applied cosmetic, the magnesium salt may absorb moisture in air to dissolve in the moisture, thereby forming a liquid. Thus, the magnesium salt is thought to become a hydrated-film-like state rather than a crystallized film. As a result, most or all of the compacted hyaluronic acid may efficiently penetrate into the skin without being immobilized in the film, and hence a better moisture-retaining property may be achieved compared to cases where a sodium salt or the like is used.

### <<Cosmetic>>

The cosmetic of the present disclosure comprises: hyaluronic acid; and a magnesium salt, wherein the magnesium salt is contained at a concentration corresponding to an ionic strength of 0.01 or more, and wherein the mass ratio of the magnesium salt to the hyaluronic acid is 1.0 or more. Because of such a composition, the cosmetic of the present disclosure can provide excellent skin penetrability and an excellent moisture-retaining property, and hence can contribute to improvement of skin problems such as wrinkles.

### <Hyaluronic Acid>

The hyaluronic acid of the present disclosure is not limited. In general, hyaluronic acid means a linear polymer in which N-acetyl-D-glucosamine residues and D-glucuronic acid residues are alternately linked, and such hyaluronic acid can be obtained, for example, by isolation extraction from a cockscomb or another animal tissue, or by fermentation using a microorganism such as the genus Streptococcus.

The hyaluronic acid may be a derivative of hyaluronic acid. Examples of the hyaluronic acid derivative that may be used include hyaluronic acid salts, more specifically, metal salts of hyaluronic acid, such as the sodium salt of hyaluronic acid, the potassium salt of hyaluronic acid, the magnesium salt of hyaluronic acid, the calcium salt of hyaluronic acid, and the aluminum salt of hyaluronic acid; and also include derivatives of hyaluronic acid and hyaluronic acid salts obtained by etherification, esterification, amidation, acetylation, acetalization, or ketalization of a hydroxyl group, carboxyl group, or the like of hyaluronic acid. Here, “hyaluronic acid” in the present disclosure may encompass the concept of hyaluronic acid and derivatives thereof.

The weight average molecular weight of the hyaluronic acid is not limited, and may be, for example, 10,000,000 or less. Not only hyaluronic acid, but also other low-molecular-weight drugs are generally considered to easily penetrate into the skin. Since a low-molecular-weight hyaluronic acid hardly stays in the skin, and its water-retaining performance is lower than that of a high-molecular-weight hyaluronic acid, it may be difficult for the low-molecular-weight hyaluronic acid to maintain the moisture-retaining effect in the skin over a long period of time. Therefore, for the purpose of maintaining the moisture-retaining effect in the skin over a long period of time, it is advantageous to use a high-molecular-weight hyaluronic acid. From the viewpoint of the water-retaining performance of the hyaluronic acid and the compaction of the hyaluronic acid, the weight average molecular weight of the hyaluronic acid may be, for example, 500 or more, 1,000 or more, 5,000 or more, 10,000 or more, 50,000 or more, 100,000 or more, 300,000 or more, 500,000 or more, 800,000 or more, or 1,000,000 or more, and may be, for example, 10,000,000 or less, 8,000,000 or less, 5,000,000 or less, 3,000,000 or less, 2,000,000 or less, or 1,500,000 or less. Here, the weight-average molecular weight means the weight-average molecular weight in terms of polystyrene in gel permeation chromatography measurement.

As the hyaluronic acid, hyaluronic acid and its derivatives may be used individually, or as a combination of two or more thereof. The hyaluronic acid and/or its derivative(s) used may have either the same molecular weight or different molecular weights.

A commercially available hyaluronic acid may be used. Examples of the commercially available hyaluronic acid include hyaluronic acid HA-LQ (manufactured by Kewpie Corporation), hyaluronic acid FCH (manufactured by Kikkoman Biochemical Company), hyaluronic acid IW120 (manufactured by Iwaki & Co., Ltd.), and sodium bio-hyaluronate HA12N (manufactured by Shiseido Co., Ltd.).

The hyaluronic acid of the present disclosure is thought to be shrunk due to the electrostatic shielding effect based on the magnesium salt, and the action of hydrogen bonding, to exhibit a form in which single hyaluronic acid molecules or a plurality of hyaluronic acid molecules entangled with each other are aggregated (such a form may be referred to as “hyaluronic acid aggregated/shrunken product”). Since a hyaluronic acid in such a form can be prepared without using the support materials as described in PTLs 1 and 2, the content ratio of hyaluronic acid in the hyaluronic acid aggregated/shrunken product can be theoretically 100% by mass. Thus, the hyaluronic acid aggregated/shrunken product of the present disclosure can be formed using hyaluronic acid molecules alone as a polymer component.

However, the hyaluronic acid aggregated/shrunken product of the present disclosure may also contain a polymer component other than hyaluronic acid as long as the skin penetrability, the moisture-retaining performance, and the like are not deteriorated. The content ratio of the other polymer component may be, for example, 20% by mass or less, 10% by mass or less, 5% by mass or less, 3% by mass or less, 1% by mass or less, 0.5% by mass or less, 0.1% by mass or less, 0.05% by mass or less, or 0.01% by mass or less with respect to the total amount of the polymers contained in the hyaluronic acid aggregated/shrunken product. Thus, in the present disclosure, the “hyaluronic acid aggregated/shrunken product” may mean a product containing a hyaluronic acid component at high concentration, and does not encompass particles in which the ratio of the hyaluronic acid component is 50% by mass or less such as those described in PTLs 1 and 2. Here, the content ratio of hyaluronic acid in the hyaluronic acid aggregated/shrunken product can be measured using, for example, the ELISA method.

In some embodiments, the amount of the magnesium salt added and/or the like may be adjusted to form the hyaluronic acid aggregated/shrunken product into a particle form having a shape of threaded ball (this form may be further referred to as “hyaluronic acid particle”) that can be measured by the dynamic light scattering method. The penetration of hyaluronic acid particles into the skin or body hair varies among individuals depending on, for example, the state of the stratum corneum, the state of the hair, and the number or size of the pores. However, in cases where the particles have an average particle diameter of 200 nm or less, the particles can be allowed to penetrate into the skin or body hair in most cases. In some embodiments, from the viewpoint of the penetrability into the skin or body hair, ease of preparation of the particles, and the like, the average particle diameter of the hyaluronic acid particles may be, for example, 150 nm or less, 120 nm or less, or 100 nm or less, and may be 10 nm or more, 30 nm or more, or 50 nm or more. Here, the average particle diameter means the Z average particle diameter of the hyaluronic acid particles as optically measured by the dynamic light scattering method based on the assumption that the particle shape of the hyaluronic acid particles is spherical. Such an average particle diameter can be measured using, for example, a Zetasizer (manufactured by Malvern Panalytical Co., Ltd.), or a dynamic light scattering photometer DLS-8000 (manufactured by Otsuka Electronics Co., Ltd.). These measuring devices can be appropriately selected based on the overlap concentration of each hyaluronic acid. For example, a dynamic light scattering photometer DLS-8000 can be used when the hyaluronic acid concentration is less than the overlap concentration, and a Zetasizer can be used when the hyaluronic acid concentration is the overlap concentration or more. The overlap concentration can be calculated, for example, by the confocal fluorescence recovery after photobleaching method (Confocal-FRAP).

It is thought that, although hyaluronic acid particles having an average particle diameter of 200 nm or less can be easily allowed to penetrate into the skin or body hair, they can hardly stay in the skin or body hair because of their small particle diameter. In the present disclosure, in some embodiments, the average particle diameter of the hyaluronic acid particles can be adjusted to more than 200 nm by adjusting, for example, the contents of the hyaluronic acid and the magnesium salt. Hyaluronic acid particles with an average particle diameter of more than 200 nm are usually assumed to be less likely to penetrate into the skin or body hair. However, unexpectedly and surprisingly, hyaluronic acid particles having such a size prepared using a magnesium salt can be allowed to penetrate into the skin or body hair, and, as illustrated in FIG. 2, the hyaluronic acid particles that have penetrated are likely to stay in the skin or body hair, so that the moisture-retaining effect in the skin can be maintained over a long period of time.

From the viewpoint of the penetrability into the skin or body hair, retention in the skin or body hair, and the like, the average particle diameter of the hyaluronic acid particles may be more than 200 nm, 210 nm or more, 230 nm or more, 250 nm or more, 270 nm or more, or 280 nm or more, and may be 500 nm or less, 450 nm or less, 400 nm or less, 350 nm or less, or 300 nm or less.

As described above, the hyaluronic acid of the present disclosure may exhibit a particle form having a shape of threaded ball. A hyaluronic acid in such a form prepared using a magnesium salt can easily penetrate into the skin or body hair. For example, it can easily noninvasively penetrate into the skin through the stratum corneum or pores of the skin. It is generally considered that a hyaluronic acid having a relatively low molecular weight with a weight average molecular weight of less than 500 can be easily allowed to penetrate into the skin or body hair even without being formed into microparticles. However, since such a low-molecular-weight hyaluronic acid hardly stays in the skin or body hair, and its water-retaining performance is lower than that of a high-molecular-weight hyaluronic acid, it may be difficult for the low-molecular-weight hyaluronic acid to maintain the moisture-retaining effect in the skin or body hair over a long period of time. On the other hand, the hyaluronic acid particles in the present disclosure can be prepared using a hyaluronic acid having a weight average molecular weight of 500 or more, and such a hyaluronic acid can be included in the particles at a high concentration. As a result, the obtained hyaluronic acid particles tend to stay in the skin or body hair, and can maintain the moisture-retaining effect in the skin or body hair over a long period of time.

From the viewpoint of the compaction, the moisture-retaining property, the cost, and the like, the content of the hyaluronic acid in the cosmetic may be 0.005% by mass or more, 0.01% by mass or more, 0.05% by mass or more, 0.10% by mass or more, 0.15% by mass or more, 0.20% by mass or more, 0.25% by mass or more, 0.30% by mass or more, 0.35% by mass or more, 0.40% by mass or more, or 0.45% by mass or more, and may be 5.0% or less, 4.0% or less, 3.0% or less, 2.0% or less, 1.0% by mass or less, 0.90% by mass or less, 0.80% by mass or less, 0.70% by mass or less, 0.60% by mass or less, 0.50% by mass or less, or 0.45% by mass or less, with respect to the total amount of the cosmetic. The hyaluronic acid contained in the cosmetic of the present disclosure can exhibit a sufficient moisture-retaining effect even in cases where the hyaluronic acid content in the cosmetic is relatively low. This is because the content ratio of hyaluronic acid in the hyaluronic acid aggregated/shrunken product is high, and a high-molecular-weight hyaluronic acid can be used to take advantage of its high moisture-retaining property.

### <Magnesium Salt>

The cosmetic of the present disclosure comprises a magnesium salt. Examples of the magnesium salt include magnesium salts constituted by at least one selected from inorganic salts and organic acid salts. Taking the use as a cosmetic into account, the salt, among inorganic salts and organic acid salts, is preferably a salt which hardly adversely affects the skin or body hair. Here, the term “inorganic salt” means a salt comprising only inorganic components, and this can also be said to be a salt formed by ions generated from an inorganic acid and an inorganic base. The term “organic acid salt” means a salt formed by an organic acid and a metal ion bound to each other. The salt in the cosmetic is generally present in the form of ions derived from the salt. Thus, in the present disclosure, for example, the term “cosmetic containing a salt” means that a magnesium salt is contained in the form of such ions.

Examples of the magnesium salts in the form of an inorganic salt include magnesium nitrate, magnesium sulfate, and magnesium chloride. These salts may be used individually, or as a combination of two or more thereof.

Examples of the organic acid salts include citrate, acetate, lactate, tartrate, succinate, malate, glycolate, salicylate, and pyrrolidone carboxylate. Specific examples of the magnesium salts in the form of an organic acid salt include salts in which an organic acid such as citric acid, acetic acid, lactic acid, tartaric acid, succinic acid, malic acid, glycolic acid, salicylic acid, or pyrrolidone carboxylic acid is bound to a magnesium ion. These salts may be used individually, or as a combination of two or more thereof.

From the viewpoint of the penetrability into the body surface or body hair, maintenance of the moisture-retaining effect, and the like, the magnesium salt is preferably a magnesium salt in the form of an inorganic salt, more preferably magnesium chloride.

Taking into account, for example, the balance regarding the degree of compaction of hyaluronic acid, the penetrability into the body surface or body hair, and/or maintenance of the moisture-retaining effect, the content of the magnesium salt in the cosmetic may be 0.01% by mass or more, 0.05% by mass or more, 0.10% by mass or more, 0.15% by mass or more, 0.20% by mass or more, 0.25% by mass or more, 0.30% by mass or more, 0.35% by mass or more, 0.40% by mass or more, 0.45% by mass or more, 0.50% by mass or more, 0.55% by mass or more, 0.60% by mass or more, 0.65% by mass or more, or 0.70% by mass or more, and may be 5.0% by mass or less, 4.0% by mass or less, 3.0% by mass or less, 2.0% by mass or less, 1.9% by mass or less, 1.8% by mass or less, 1.7% by mass or less, 1.6% by mass or less, or 1.5% by mass or less, with respect to the total amount of the cosmetic. In cases where the magnesium salt is in the form of a hydrate, the content of the magnesium salt does not include the amount of water in the hydrate.

The content of the magnesium salt in the cosmetic can be defined also as the ionic strength of the magnesium salt. Taking into account, for example, the balance regarding the degree of compaction of hyaluronic acid, the penetrability into the body surface or body hair, and/or maintenance of the moisture-retaining effect, the ionic strength of the magnesium salt is preferably 0.01 or more, 0.03 or more, 0.05 or more, 0.07 or more, 0.10 or more, more than 0.10, 0.12 or more, 0.15 or more, 0.17 or more, 0.20 or more, 0.22 or more, or 0.25 or more. Regarding the upper limit, the ionic strength may be, for example, 5.0 or less, 4.0 or less, 3.0 or less, 2.0 or less, 1.0 or less, 0.70 or less, or 0.50 or less.

Here, for example, when the cosmetic is prepared by adding a magnesium salt to a buffer, the content or ionic strength of the magnesium salt is calculated based on all magnesium salt components including the magnesium salt component(s) contained in the buffer itself and the magnesium salt component(s) separately added to the buffer.

In the cosmetic of the present disclosure, the magnesium salt is contained such that its concentration achieves the ionic strength described above, and such that the mass ratio of the magnesium salt to the hyaluronic acid is 1.0 or more. Thus, since the magnesium salt, which may exhibit deliquescence, is included at a well-balanced prescribed ratio, the cosmetic of the present disclosure is less likely to crystallize on the surface of the skin or the like, and can provide better skin penetrability and a better moisture-retaining property compared to cosmetics containing hyaluronic acid compacted using other salts, for example, sodium chloride, such as the cosmetic described in PTL 3.

Taking into account, for example, the balance regarding the degree of compaction of hyaluronic acid, the penetrability into the body surface or body hair, and/or maintenance of the moisture-retaining effect, the mass ratio of the magnesium salt to the hyaluronic acid is preferably 1.0 or more, 1.1 or more, 1.2 or more, 1.3 or more, 1.4 or more, 1.5 or more, 1.6 or more, 1.7 or more, 1.8 or more, 1.9 or more, or 2.0 or more. Regarding the upper limit, the mass ratio may be, for example, 10 or less, 9.0 or less, 8.0 or less, 7.0 or less, 6.0 or less, 5.0 or less, 4.0 or less, or 3.0 or less.

### <Aqueous Medium>

The cosmetic of the present disclosure may typically contain an aqueous medium. The aqueous medium is not limited, and aqueous media used in cosmetics, quasi-drugs, and the like may be used. Examples of the aqueous media that may be used include ion-exchanged water, distilled water, ultrapure water, tap water, and buffers. These aqueous media may be used individually, or as a combination of two or more thereof.

Examples of the buffers include citrate buffer, lactate buffer, phosphate buffer, acetate buffer, tartrate buffer, borate buffer, and Tris buffer. From the viewpoint of securing a high buffer capacity, citrate buffer, lactate buffer, and phosphate buffer are preferred. Citrate buffer is more preferred.

The pH of the buffer may be 7.0 or less, 6.8 or less, or 6.5 or less. There is no lower limit value of the pH of the buffer. From the viewpoint of, for example, reducing irritation to the skin, the pH is preferably 4.5 or more, 5.5 or more, or 6.0 or more.

### <Optional Components>

In the cosmetic of the present disclosure, various components may be included, when appropriate, within a range that does not adversely affect the effect of the present invention. Examples of such components include salts other than magnesium salts; humectants; skin or hair nutrients; vitamins; water-soluble agents applicable to pharmaceuticals, quasi-drugs, cosmetics, and the like; ultraviolet absorbers; antioxidants; preservatives; antioxidant aids; thickeners; surfactants (for example, anionic surfactants, cationic surfactants, nonionic surfactants, and amphoteric surfactants), pigments; dyes; colorants; and fragrances. These optional components may be used individually, or as a combination of two or more thereof.

<Salt Other than Magnesium Salts>

The cosmetic of the present disclosure may comprise a salt other than magnesium salts. The content of the other salt in the cosmetic may be, for example, less than 0.50% by mass, 0.40% by mass or less, 0.30% by mass or less, 0.20% by mass or less, 0.10% by mass or less, less than 0.10% by mass, 0.05% by mass or less, or 0.01% by mass or less. From the viewpoint of the penetrability into the body surface or body hair, maintenance of the moisture-retaining effect, and the like, salts other than magnesium salts are preferably not contained in the cosmetic. Here, the “salts other than magnesium salts” in the present disclosure do not include ionic surfactants and hyaluronic acid salts.

### (Amphoteric Surfactant)

The cosmetic of the present disclosure may comprise an amphoteric surfactant. The content of the amphoteric surfactant in the cosmetic may be, for example, less than 0.01% by mass, 0.005% by mass or less, or 0.001% by mass or less. The amphoteric surfactant does not need to be included in the cosmetic.

### <<Formulation and Product Form of Cosmetic>>

The formulation of the cosmetic of the present disclosure is not limited, and examples of the formulation include a liquid, an emulsion, a cream, a gel, a mist, a spray, an aerosol, and a mousse. These may be in the form of a single-phase system, or may be in the form of a two-phase system of a non-emulsified or emulsified, oil-in-water or water-in-oil type. The term “spray” in the present disclosure may encompass mist-type sprays, aerosol-type sprays, and the like.

The product form of the cosmetic of the present disclosure is also not limited, and examples of the product form include: skin care cosmetics such as emulsions, creams, face oils, body oils, and beauty essences; make-up cosmetics such as foundations, make-up bases, lipsticks, blushers, eye shadows, mascaras, and mascara bases; sunscreen cosmetics; body cosmetics; aromatic cosmetics; skin-washing agents such as make-up removers, facial cleansers, and body shampoos; and hair cosmetics such as hair sprays, hair creams, hair lotions, hair conditioners, and shampoos. In particular, the cosmetic can be advantageously used as a leave-on type cosmetic that focuses on penetration of hyaluronic acid into the skin or the like, and that can be used without rinsing.

In some embodiments, the cosmetics of the present disclosure can be prepared as a cosmetic (such as a beauty essence) based on a solubilized system or a microemulsion system. Such a cosmetic may have a semi-transparent or transparent appearance. Such an appearance can be evaluated, for example, based on the L value, which is calculated using a color difference meter such as COLOR-EYE 7000A (manufactured by Gretag Macbeth). The closer the L value to 100, the higher the transparency. In some embodiments, the cosmetic of the present disclosure may have an L value of 70 or more, 75 or more, 80 or more, 85 or more, 90 or more, 92 or more, or 95 or more, and 100 or less, or less than 100.

In some embodiments, the cosmetic of the present disclosure does not contain a second agent containing a chelating agent that is used to swell the above-described hyaluronic acid after application of the hyaluronic acid to the skin.

### <<Preparation Method for Cosmetic>>

The cosmetic of the present disclosure can be prepared, for example, using the following method. As materials such as the hyaluronic acid, magnesium salt, water, and optional components that may be used in the preparation method for the cosmetic, the above-described materials may be used.

The cosmetic can be prepared by adding a magnesium salt to water or a buffer to prepare a solution, adding hyaluronic acid to the resulting solution, and then dissolving the hyaluronic acid with stirring and mixing.

Alternatively, the cosmetic can be prepared by adding hyaluronic acid to water or a buffer, dissolving the hyaluronic acid with stirring and mixing, and then adding a magnesium salt to the resulting solution.

In cases where the buffer itself exhibits the above-described magnesium salt concentration or ionic strength by the action of a magnesium salt contained in the buffer, the addition of the magnesium salt can be omitted.

In cases where the optional components described above are to be included, such optional components may be added before the compaction of the hyaluronic acid using the magnesium salt, but the optional components are preferably added after the preparation of the compacted hyaluronic acid so as not to affect the compacted hyaluronic acid.

### <<Application Site of Cosmetic>>

The cosmetic of the present disclosure is applicable to any part of the body. For example, the cosmetic may be applied to any part on the skin surface (body surface) or on the body hair (hair). More specifically, the cosmetic of the present disclosure may be applied, when appropriate, to the skin surface of the face (lips, eyes, eyelids, cheeks, forehead, glabella, nose, or the like), head (scalp), ears, hands, arms, neck, legs, feet, chest, abdomen, back, or the like, or to a body hair such as scalp hair, eyebrows, lashes, or mustache. Here, the skin also includes nails and the like formed by hardening of the corneum of the skin epidermis. In the present disclosure, “body hair” has the same meaning as “hair”, and encompasses all kinds of hair on the body. Specific examples of the body hair (hair) include scalp hair, eyelashes, eyebrows, and beards.

Thus, the cosmetic of the present disclosure can be suitably used as a cosmetic for the body surface or body hair. Further, since the cosmetic of the present disclosure allows penetration of hyaluronic acid into the body surface or body hair, the cosmetic can be suitably used as a cosmetic for penetration of hyaluronic acid into the body surface or body hair.

<<Beauty method Using Cosmetic>>

The beauty method using the cosmetic of the present disclosure comprises: applying the cosmetic described above to the body surface or body hair. In the present disclosure, “beauty method” means a method of beautifying a body surface or body hair by achieving a beautiful and orderly condition of the body surface or body hair by application of the cosmetic of the present disclosure to the body surface or body hair. The method is thus different from a method of operation, treatment, or diagnosis of a human.

In general, one unknowingly suffers from deprivation of moisture from skin or body hair when the skin or body hair is exposed to dryness, which deprivation leads to a state where the water content on the surface of the skin or body hair cannot be maintained. For example, when the moisture on the skin surface is insufficient, a moisturizing component to be produced by the skin itself (Natural Moisturizing Factor (NMF)) cannot be successfully produced. As a result, the barrier function and the moisturizing function on the skin surface deteriorate, and the skin tends to be damaged. This has been thought to result in loss of moisture, formation of wrinkles, skin roughness, and the like. Further, it has been thought that dryness of hairs due to loss of moisture causes formation of split ends, hair breakage, easy spreading of the hairs due to static electricity, and the like.

Since the cosmetic of the present disclosure allows favorable penetration of compacted hyaluronic acid into the skin or body hair, the moisture in the skin or body hair can be favorably retained. As a result, for example, the function that allows the skin itself to generate the moisturizing component can be improved, and moreover, poor turnover in the stratum corneum can be improved, or moisture of hair can be improved. Therefore, troubles such as skin roughness, or formation of split ends or hair breakage can be reduced to enhance the beauty effect.

The means of application of the cosmetic to the body surface or body hair is not limited. For example, the application may be performed by applying the cosmetic onto the body surface or body hair. Regarding the means of applying the cosmetic onto the body surface or body hair, the application may be carried out by spraying the cosmetic onto the body surface or body hair using a spray container to which the cosmetic has been fed. Alternatively, the application may be carried out by feeding the cosmetic to a container having no spray function, collecting an appropriate amount of the cosmetic from the container onto a finger, palm, or the like, and then spreading the cosmetic on the body surface or body hair.

From the viewpoint of enhancing the beauty effect, the application of the cosmetic to the body surface or body hair is preferably carried out for 2 days or more, 5 days or more, 7 days or more, 10 days or more, 15 days or more, 20 days or more, or 30 days or more. From the viewpoint of even enhancing the beauty effect, the application of the cosmetic to the body surface or body hair is more preferably carried out continuously for 2 days or more, 5 days or more, 7 days or more, 10 days or more, 15 days or more, 20 days or more, or 30 days or more. Here, the continuous application may be referred to as “continuous use”.

### EXAMPLES

Test Examples and Examples are described below to illustrate the present invention in more detail. However, the present invention is not limited thereto. Unless otherwise specified, each content is hereinafter expressed in % by mass.

### Test Examples 1 to 5 and Reference Test Example 1

### <Evaluation of Cosmetics>

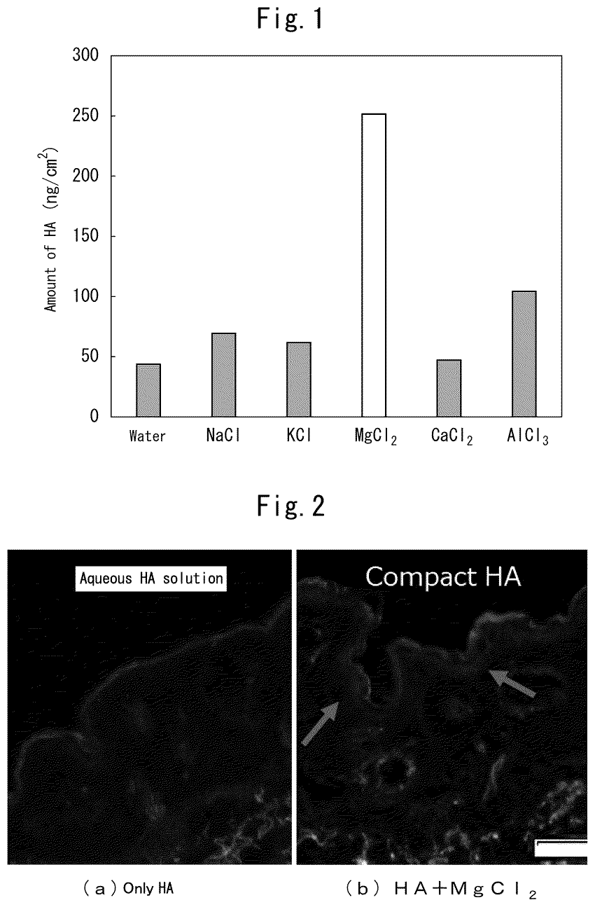

The evaluations described below were carried out using test samples of cosmetics obtained by the following production methods. The results are summarized in Tables 1 to 4 and FIGS. 1 to 3. In the figures, “HA” means hyaluronic acid.

(Penetrability Test 1: Evaluation of Amount of Hyaluronic Acid that has Penetrated into Human Skin)

The penetrability of hyaluronic acid was evaluated from the amount of hyaluronic acid recovered from stratum corneum peeled off using tapes of the D-squame (trademark) disc according to (1) to (5) in the following procedure.
(1) Human skin (60s) was placed in a Diffusion Cell Array System (manufactured by Introtec Co., Ltd.), and degassed phosphate buffered saline (PBS) was filled into the receiver, to hydrate the skin for 1 hour.
(2) After confirming that the skin surface temperature was 32° C., a test sample of a cosmetic was applied to the skin at 10 μL/cm².
(3) Six hours later, the skin surface (stratum corneum) was wiped five times with a cotton swab impregnated with soap solution, and then five times with a cotton swab impregnated with MilliQ (trademark) water (ultrapure water), to clean the stratum corneum.
(4) The cleaned stratum corneum was peeled off using tapes of the D-squame (trademark) disc, wherein peeling-off was repeated 20 times using tapes. The 6th to 10th tapes were immersed in 2.5 mL of MilliQ (trademark) water supplemented with 5% methanol, and then subjected to sonication for 20 minutes to extract hyaluronic acid.
(5) Using a Hyaluronan Quantification Kit (manufactured by Cosmo Bio Co., Ltd.), the resulting extract was analyzed by the ELISA method to quantify the amount of hyaluronic acid in the extract.
   (Penetrability Test 2: Fluorescence Microscopic Evaluation of Penetrability of Hyaluronic Acid that has Penetrated into Human Skin)

Hyaluronic acid that has penetrated into the skin was directly observed according to (1) to (5) in the following procedure.
(1) Human skin (60s) was placed in a Diffusion Cell Array System (manufactured by Introtec Co., Ltd.), and degassed PBS was filled into the receiver, to hydrate the skin for 1 hour.
(2) After confirming that the skin surface temperature was 32° C., a test sample of a cosmetic was applied to the skin at 10 μL/cm².
(3) Six hours later, the skin surface (stratum corneum) was wiped with a cotton swab impregnated with MilliQ (trademark) water (ultrapure water).
(4) The skin was excised, and the excised skin was embedded in the O. C. T (Optimal Cutting Temperature) compound, followed by preparation of sections in a cryostat (CM3050S, manufactured by Leica Microsystems, Inc.).
(5) The prepared sections were observed under a fluorescence microscope (BX51, manufactured by Olympus Corporation).

Fluorescein amine (FA)-labeled hyaluronic acid (HA) was prepared by application of the fluorescent group fluorescein amine (FA) as follows.

In 500 mL of methanol (manufactured by Nacalai Tesque, Inc.), 10 g of Bio-Hyaro 12 (manufactured by Shiseido Co., Ltd.) was uniformly dispersed, and then 500 mL of water was added thereto to allow complete dissolution. To the resulting solution, a solution containing 100 mg of 5-aminofluorescein (manufactured by Tokyo Chemical Industry Co., Ltd.) in methanol and a solution containing 104 mg of the dehydration-condensation agent DMT-MM (manufactured by Kokusan Chemical Co., Ltd.) in methanol were added with stirring, to obtain an FA-labeled HA. Further, the obtained FA-labeled HA was dialyzed with purified water for 1 week, and then freeze-dried to obtain about 6 g of a purified FA-labeled HA. The FA-labeled HA showed a maximum absorption wavelength of 494 nm and a maximum fluorescence wavelength of 521 nm as measured using a spectrofluorometer (manufactured by JASCO Corporation).

(Penetrability Test 3: Evaluation of Amount of Hyaluronic Acid that has Penetrated into Human Skin)

The penetrability of hyaluronic acid was evaluated from the amount of hyaluronic acid recovered from stratum corneum peeled off using tapes of the D-squame (trademark) disc according to (1) to (5) in the following procedure.
(1) Human skin (47 years old, male) was placed in a Diffusion Cell Array System (manufactured by Introtec Co., Ltd.), and degassed PBS was filled into the receiver, to hydrate the skin for 1 hour.
(2) After confirming that the skin surface temperature was 32° C., a test sample of a cosmetic was applied to the skin at 10 μL/cm².
(3) Six hours later, the skin surface (stratum corneum) was wiped five times with a cotton swab impregnated with soap solution, and then five times with a cotton swab impregnated with MilliQ (trademark) water (ultrapure water), to clean the stratum corneum.
(4) The cleaned stratum corneum was peeled off using tapes of the D-squame (trademark) disc, wherein peeling-off was repeated 20 times using tapes. Each of the 1st to 5th tapes, the 6th to 10th tapes, and the 11th to 15th tapes was immersed in 2.5 mL of MilliQ (trademark) water supplemented with 5% methanol, and then subjected to sonication for 20 minutes to extract hyaluronic acid.
(5) Using a QnE Hyaluronic Acid (HA) ELISA Assay Kit (manufactured by Funakoshi Co., Ltd.), the resulting extract was analyzed by the ELISA method to quantify the amount of hyaluronic acid in the extract.
   (Penetrability Test 4: Evaluation of Amount of Hyaluronic Acid that has Penetrated into Human Skin)

According to (1) and (2) in the following procedure, the penetrability of hyaluronic acid was evaluated from the amount of hyaluronic acid that has penetrated from a pseudo-skin into a medium.
(1) Five milliliters of a medium (EPI-100-LLMM, manufactured by MATTEK) warmed at 37° C. was dispensed into a 12-well plate, and then a pseudo-skin membrane (Melano Derm MEL-300 Kit, Asian Donor (MEL-300-A), manufactured by MATTEK) was transferred to the 12-well plate, followed by applying 25 μL of a test sample to the upper side of the pseudo-skin membrane and performing culture in a CO₂ incubator (37° C., 5% CO₂, wet) for one day.
(2) After collecting 1 mL of a sample from the cultured medium, the sample was analyzed by the ELISA method to quantify the amount of hyaluronic acid in the medium.
   (Penetrability Test 5: Evaluation of Amount of Hyaluronic Acid that has Penetrated into Human Skin after One Month of Continuous Use)

The penetrability of hyaluronic acid after one month of continuous use was evaluated from the amount of hyaluronic acid recovered from stratum corneum peeled off using tapes of the D-squame (trademark) disc according to (1) to (3) in the following procedure.
(1) For 12 healthy male panelists in their 20s or 30s, the inner side of a forearm was cleaned with soap, and then 1 mL of each cosmetic sample was applied to the cleaned skin.
(2) The operation of (1) was carried out once every day continuously for one month, and then stratum corneum of the skin to which the sample was applied was peeled off using tapes of the D-squame (trademark) disc, wherein peeling-off was repeated five times using tapes. The 3rd to 5th tapes were immersed in MilliQ (trademark) water supplemented with 5% methanol, and then subjected to sonication for 20 minutes to extract hyaluronic acid.
(3) Using a Hyaluronan Quantification Kit (manufactured by Cosmo Bio Co., Ltd.), the resulting extract was analyzed by the ELISA method to quantify the amount of hyaluronic acid in the extract.

### (Evaluation of Average Particle Diameter of Compacted Hyaluronic Acid)

According to (1) and (2) in the following procedure, the average particle diameter of compacted hyaluronic acid in a test sample was evaluated.
(1) A test sample was filtered through a 1-μm membrane filter.
(2) The Z-average particle diameter of the filtered sample was determined by the dynamic light scattering method using a Zetasizer (manufactured by Malvern Panalytical Co., Ltd.).

### <Test Example 1: Effects of Various Salts)

In Test Example 1, based on the penetrability test 1 described above, the effects of various salts that were added together with hyaluronic acid were studied. The results are shown in Table 1 and FIG. 1.

### Comparative Example 1

Hyaluronic acid (manufactured by Shiseido Co., Ltd., Bio-Hyaro 12; weight average molecular weight, 1,200,000) was added to ion-exchanged water to a content of 0.50% by mass, and the resulting mixture was mixed by stirring using a vortex mixer, to prepare Composition A. After dissolution of the hyaluronic acid, sodium chloride (weight average molecular weight (Mw): 58.4; manufactured by Junsei Chemical Co., Ltd.) was added to Composition A to the concentration corresponding to a metal ion equivalent of 0.14 Eq (0.82% by mass), and the resulting mixture was mixed by stirring using a vortex mixer, to prepare a test sample.

### Comparative Examples 2 to 4 and Example 1

The test samples of Comparative Examples 2 to 4 and Example 1 were prepared in the same manner as in Comparative Example 1 except that the salts in Table 1 were used.

### Reference Example 1

As the test sample (control sample) of Reference Example 1, Composition A in Comparative Example 1 without addition of the salt was used.

TABLE 1

| | Reference | Comparative | Comparative | Example | Comparative | Comparative |
|---|---|---|---|---|---|---|
| | Example 1 | Example 1 | Example 2 | 1 | Example 3 | Example 4 |
| Hyaluronic acid | 0.50 | 0.50 | 0.50 | 0.50 | 0.50 | 0.50 |
| NaCl¹⁾ | — | 0.82 | — | — | — | — |
| KCl²⁾ | — | — | 1.04 | — | — | — |
| MgCl₂•6H₂O³⁾ | — | — | — | 1.42 | — | — |
| CaCl₂⁴⁾ | — | — | — | — | 1.03 | — |
| AlCl₃⁵⁾ | — | — | — | — | — | 1.13 |
| Water | Remainder | Remainder | Remainder | Remainder | Remainder | Remainder |
| Total | 100 | 100 | 100 | 100 | 100 | 100 |
| Amount of hyaluronic | 43.7 | 69.4 | 61.8 | 251.5 | 47.2 | 104.2 |
| acid (ng/cm²) | | | | | | |

¹⁾Sodium chloride: manufactured by Junsei Chemical Co., Ltd. (Mw: 58.4)
²⁾Potassium chloride: manufactured by Fujifilm Wako Pure Chemical Corporation (Mw: 74.6)
³⁾Magnesium chloride hexahydrate: manufactured by Fujifilm Wako Pure Chemical Corporation (Mw: 203.3)
⁴⁾Calcium chloride: manufactured by Nacalai Tesque, Inc. (Mw: 147.0)
⁵⁾Aluminum chloride: manufactured by Nacalai Tesque, Inc. (Mw: 241.4)

### (Results)

As is evident from Table 1 and FIG. 1, even in the cases where a salt other than a magnesium salt was used, the amount of hyaluronic acid increased compared to Reference Example 1, which contains no salt, indicating improvement of the penetrability of hyaluronic acid into the skin. However, it could be confirmed that use of a magnesium salt leads to remarkable improvement of the penetrability of hyaluronic acid into the skin even compared to the cases where the other salts were used.

### Test Example 2: Direct Observation of Hyaluronic Acid that has Penetrated into Skin

In Test Example 2, based on the penetrability test 2 described above, the degree of penetration of hyaluronic acid into the skin was studied for a case where a magnesium salt was used. The result is shown in FIG. 2.

### (Test Samples)

The test samples of Reference Example 1 and Example 1 in Test Example 1 were used.

### (Result)

As can be seen from FIG. 2, accumulation of more hyaluronic acid was found in stratum corneum portions (the portions indicated by the arrowheads) in the case where the aqueous solution containing hyaluronic acid prepared using the magnesium salt was applied to the skin (FIG. 2(b)) than in the case where the aqueous solution containing hyaluronic acid for which the magnesium salt was not used was applied to the skin (FIG. 2(a)). From this result, it was found that a hyaluronic acid prepared using a prescribed amount of magnesium salt can enhance the moisture-retaining effect inside the skin since the hyaluronic acid is likely to penetrate into the skin and stay near the stratum corneum.

### Test Example 3: Effect of Content Ratio of Magnesium Salt 1

In Test Example 3, based on the penetrability test 3 described above, the effect of the content ratio of the magnesium salt was studied. The results are shown in Tables 2 to 4. Here, in Tables 2 to 4, “1 to 5”, “6 to 10”, and “11 to 15” mean the amounts of hyaluronic acid collected by the 1st to 5th tapes, the 6th to 10th tapes, and the 11th to 15th tapes. In the present disclosure, “amount in terms of MgCl₂” means the amount calculated by excluding the amount of water in the hydrate from the content of magnesium chloride hexahydrate (MgCl₂·6H₂O). When “amount in terms of MgCl₂” is described in a table, the total amount of the mixture composition is the total amount of components excluding the amount in terms of MgCl₂. In Reference Example 3 and Comparative Example 8, shown in Table 3, “human skin (47 years old, male)” in (1) and “6 hours” in (3) of the penetrability test 3 described above were replaced with “human skin (38 years old, female)” and “4 hours”, respectively. In Reference Example 4 and Example 4, shown in Table 4, “human skin (47 years old, male)” in (1) and “6 hours” in (3) of the penetrability test 3 described above were replaced with “human skin (34 years old, female)” and “4 hours”, respectively.

### Comparative Example 5

Hyaluronic acid (manufactured by Shiseido Co., Ltd., Bio-Hyaro 12; weight average molecular weight, 1,200,000) was added to ion-exchanged water to a content of 0.50% by mass, and the resulting mixture was mixed by stirring using a vortex mixer, to prepare Composition B. After dissolution of the hyaluronic acid, MgCl₂·6H₂O was added at 0.17% by mass to Composition B, and the resulting mixture was mixed by stirring using a vortex mixer, to prepare a test sample.

### Comparative Examples 6 and 7, and Examples 2 and 3

The test samples of Comparative Examples 6 and 7, and Examples 2 and 3 were prepared in the same manner as in Comparative Example 5 except that the value of the amount of MgCl₂·6H₂O added was as shown in Table 2.

### Reference Example 2

As the test sample (control sample) of Reference Example 2, Composition B in Comparative Example 5 without addition of the salt was used.

### Comparative Example 8

Hyaluronic acid (manufactured by Shiseido Co., Ltd., Bio-Hyaro 12; weight average molecular weight, 1,200,000) was added to ion-exchanged water to a content of 0.10% by mass, and the resulting mixture was mixed by stirring using a vortex mixer, to prepare Composition B. After dissolution of the hyaluronic acid, MgCl₂·6H₂O was added at 0.17% by mass to Composition B, and the resulting mixture was mixed by stirring using a vortex mixer, to prepare a test sample.

### Reference Example 3

As the test sample (control sample) of Reference Example 3, Composition B in Comparative Example 8 without addition of the salt was used.

### Example 4

Hyaluronic acid (manufactured by Shiseido Co., Ltd., Bio-Hyaro 12; weight average molecular weight, 1,200,000) was added to ion-exchanged water to a content of 0.10% by mass, and the resulting mixture was mixed by stirring using a vortex mixer, to prepare Composition B. After dissolution of the hyaluronic acid, MgCl₂·6H₂O was added at 1.00% by mass to Composition B, and the resulting mixture was mixed by stirring using a vortex mixer, to prepare a test sample.

### Reference Example 4

As the test sample (control sample) of Reference Example 4, the test sample of Reference Example 3 was used.

TABLE 2

| | | Reference | Comparative | Comparative | Comparative | Example | Example |
|---|---|---|---|---|---|---|---|
| | | Example 2 | Example 5 | Example 6 | Example 7 | 2 | 3 |
| Hyaluronic acid | | 0.50 | 0.50 | 0.50 | 0.50 | 0.50 | 0.50 |
| MgCl₂•6H₂O | | — | 0.17 | 0.39 | 0.68 | 1.36 | 2.71 |
| Amount in terms of MgCl₂ | | — | 0.08 | 0.18 | 0.32 | 0.64 | 1.27 |
| Water | | Remainder | Remainder | Remainder | Remainder | Remainder | Remainder |
| Total | | 100 | 100 | 100 | 100 | 100 | 100 |
| Ionic strength | | — | 0.03 | 0.06 | 0.10 | 0.20 | 0.40 |
| Mg salt/hyaluronic acid | | — | 0.2 | 0.4 | 0.6 | 1.3 | 2.5 |
| (mass ratio) | | | | | | | |
| Amount of hyaluronic | 1 to 5 | 33.7 | 45.7 | 33.9 | 28.6 | 67.9 | 83.3 |
| acid (ng/cm²) | 6 to 10 | 11.4 | 24.2 | 16.1 | 14.4 | 30.7 | 29.6 |
| | 11 to 15 | 7.9 | 12.7 | 12.6 | 14.3 | 24.9 | 25.3 |
| | Total | 53.0 | 82.6 | 62.7 | 57.3 | 123.5 | 138.3 |

TABLE 3

| | | Reference | Comparative |
|---|---|---|---|
| | | Example 3 | Example 8 |
| Hyaluronic acid | | 0.10 | 0.10 |
| MgCl₂•6H₂O | | — | 0.17 |
| Amount in terms of MgCl₂ | | — | 0.08 |
| Water | | Remainder | Remainder |
| Total | | 100 | 100 |
| Ionic strength | | — | 0.03 |
| Mg salt/hyaluronic acid (mass ratio) | | — | 0.8 |
| Amount of hyaluronic | 1 to 5 | 8.5 | 6.5 |
| acid (ng/cm²) | 6 to 10 | 5.8 | 6.0 |
| | 11 to 15 | 4.4 | 5.3 |
| | Total | 18.6 | 17.8 |

TABLE 4

| | | Reference | |
|---|---|---|---|
| | | Example 4 | Example 4 |
| Hyaluronic acid | | 0.10 | 0.10 |
| MgCl₂•6H₂O | | — | 1.00 |
| Amount in terms of MgCl₂ | | — | 0.47 |
| Water | | Remainder | Remainder |
| Total | | 100 | 100 |
| Ionic strength | | — | 0.15 |
| Mg salt/hyaluronic acid (mass ratio) | | — | 4.7 |
| Amount of hyaluronic | 1 to 5 | 6.7 | 7.9 |
| acid (ng/cm²) | 6 to 10 | 2.9 | 6.2 |
| | 11 to 15 | 5.3 | 4.8 |
| | Total | 14.9 | 18.9 |

### (Results)

As is evident from Table 2, the amount (total amount) of hyaluronic acid was increased in Examples 2 and 3 compared to Reference Example 2 and Comparative Examples 5 to 7. It was thus found that, in cases where a magnesium salt is included at a predetermined ratio (more specifically, in cases where a magnesium salt is contained at a concentration corresponding to an ionic strength of 0.01 or more, and the mass ratio of the magnesium salt to the hyaluronic acid is 1.0 or more), the penetrability of hyaluronic acid into the skin can be largely improved. From the results in Tables 3 and 4, it could be confirmed that a similar trend can be seen even in cases where the content of hyaluronic acid is low.

The amount of hyaluronic acid shows a higher value in the 1st to 5th tapes than in the 6th to 10th tapes and the 11th to 15th tapes. From this result, it can be said that hyaluronic acid prepared using a magnesium salt tends to stay near the stratum corneum after penetration into the skin, and is hence capable of enhancing the moisture-retaining effect in the skin. This result is consistent with the result of Test Example 2.

### Test Example 4: Effect of Content Ratio of Magnesium Salt 2

In Test Example 4, based on the penetrability test 4 described above, the effect of the content ratio of the magnesium salt was studied. The results are shown in Table 5. Each value of the amount of hyaluronic acid in Table 5 is the average of data from four wells in a 12-well plate. In cases where the content of hyaluronic acid in the test sample is low (for example, about 0.10% by mass), evaluation of the effect may be impossible in the penetrability test 3 described above. Therefore, in cases where the content of hyaluronic acid is low, the evaluation is preferably carried out by the penetrability test 4.

### Reference Example 5

As the test sample, MilliQ (trademark) water (ultrapure water) was used.

### Reference Example 6

MgCl₂·6H₂O was added at 0.34% by mass to ion-exchanged water, and the resulting mixture was mixed by stirring using a vortex mixer to dissolve the MgCl₂·6H₂O, to prepare a test sample.

### Comparative Example 9

Hyaluronic acid (manufactured by Shiseido Co., Ltd., Bio-Hyaro 12; weight average molecular weight, 1,200,000) was added to ion-exchanged water to a content of 0.10% by mass, and the resulting mixture was mixed by stirring using a vortex mixer to dissolve the hyaluronic acid, to prepare a test sample.

### Example 5

Hyaluronic acid (manufactured by Shiseido Co., Ltd., Bio-Hyaro 12; weight average molecular weight, 1,200,000) was added to ion-exchanged water to a content of 0.10% by mass, and the resulting mixture was mixed by stirring using a vortex mixer, to prepare Composition C. After dissolution of the hyaluronic acid, MgCl₂·6H₂O was added at 0.34% by mass to Composition C, and the resulting mixture was mixed by stirring using a vortex mixer, to prepare a test sample.

TABLE 5

| | Reference | Reference | Comparative | Example |
|---|---|---|---|---|
| | Example 5 | Example 6 | Example 9 | 5 |
| Hyaluronic acid | — | — | 0.10 | 0.10 |
| MgCl₂•6H₂O | — | 0.34 | — | 0.34 |
| Amount in terms | — | 0.16 | — | 0.16 |
| of MgCl₂ | | | | |
| Water | 100 | Remainder | Remainder | Remainder |
| Total | 100 | 100 | 100 | 100 |
| Ionic strength | — | 0.05 | — | 0.05 |
| Mg salt/hyaluronic | — | — | — | 1.6 |
| acid (mass ratio) | | | | |
| Amount of hyaluronic | 17.4 | 20.5 | 25.9 | 35.9 |
| acid(ng/cm²) | | | | |

### (Results)

As can be seen from Table 5, it was found that, in cases where a magnesium salt is included at a prescribed ratio (more specifically, in cases where a magnesium salt is contained at a concentration corresponding to an ionic strength of 0.01 or more, and the mass ratio of the magnesium salt to the hyaluronic acid is 1.0 or more), the penetrability of hyaluronic acid into the skin can be largely improved even when the content of hyaluronic acid in the sample is low.

### Test Example 5: Effect of One Month of Continuous Use on Penetration of Hyaluronic Acid into Skin

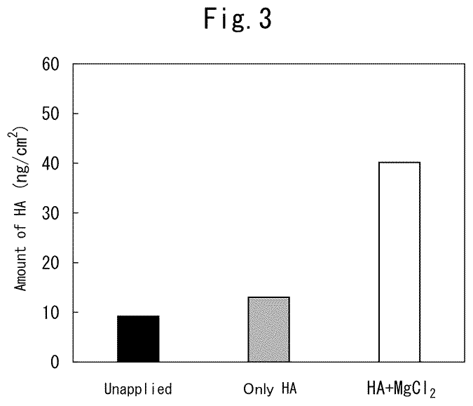

In Test Example 5, based on the penetrability test 5 described above, the effect of one month of continuous use on the penetration of hyaluronic acid into the skin was studied. The results are shown in Table 6 and FIG. 3. Each value of the amount of hyaluronic acid is the average of results from 12 panelists.

### Reference Example 7

The amount of hyaluronic acid was measured at a site where no sample was applied to the skin.

### Comparative Example 10

Hyaluronic acid (manufactured by Shiseido Co., Ltd., Bio-Hyaro 12; weight average molecular weight, 1,200,000) was added to ion-exchanged water to a content of 0.50% by mass, and the resulting mixture was mixed by stirring using a vortex mixer, to prepare Composition D. After dissolution of the hyaluronic acid, phenoxyethanol was added at 0.50% by mass, and ethanol was added at 1.00% by mass to Composition D, followed by mixing the resulting mixture by stirring using a vortex mixer, to prepare a test sample.

### Example 6

Hyaluronic acid (manufactured by Shiseido Co., Ltd., Bio-Hyaro 12; weight average molecular weight, 1,200,000) was added to ion-exchanged water to a content of 0.50% by mass, and the resulting mixture was mixed by stirring using a vortex mixer, to prepare Composition D. After dissolution of the hyaluronic acid, MgCl₂·6H₂O was added at 1.42% by mass; phenoxyethanol was added at 0.50% by mass; and ethanol was added at 1.00% by mass; to Composition D, followed by mixing the resulting mixture by stirring using a vortex mixer, to prepare a test sample.

TABLE 6

| | Reference | | |
|---|---|---|---|
| | Example 7 (no | Comparative | |
| | application) | Example 10 | Example 6 |
| Hyaluronic acid | — | 0.50 | 0.50 |
| MgCl₂•6H₂O | — | — | 1.42 |
| Amount in terms of MgCl₂ | — | — | 0.66 |
| Phenoxyethanol | — | 0.50 | 0.50 |
| Ethanol | — | 1.00 | 1.00 |
| Water | — | Remainder | Remainder |
| Total | — | 100 | 100 |
| Ionic strength | — | — | 0.21 |
| Mg salt/hyaluronic | — | — | 1.3 |
| acid (mass ratio) | | | |
| Amount of hyaluronic | 9.2 | 13.0 | 40.2 |
| acid(ng/cm²) | | | |

### (Results)

From Table 6 and FIG. 3, it was found that continuous long-term application of a composition containing hyaluronic acid prepared using a predetermined amount of magnesium salt to the skin allows favorable penetration of the hyaluronic acid into the skin.

### Reference Test Example 1: Effects of Salt Type and Hyaluronic Acid Concentration on Particle Diameter of Compacted Hyaluronic Acid

In Reference Test Example 1, the effects of the salt type and the hyaluronic acid concentration on the particle diameter of compacted hyaluronic acid were studied.

### Reference Example 8

Hyaluronic acid (manufactured by Shiseido Co., Ltd., Bio-Hyaro 12; weight average molecular weight, 1,200,000) was added to ion-exchanged water to a content of 0.50% by mass, and the resulting mixture was mixed by stirring using a vortex mixer, to prepare Composition E. After dissolution of the hyaluronic acid, sodium chloride was added to Composition E such that an ionic strength of 0.20 was achieved, and the resulting mixture was mixed by stirring using a vortex mixer, to prepare a test sample.

### Reference Example 9

A test sample of Reference Example 9 was prepared in the same manner as in Reference Example 8 except that MgCl₂·6H₂O was used instead of sodium chloride.

### Reference Example 10

The test sample of Comparative Example 9 (hyaluronic acid: 0.10% by mass) was used as the test sample of Reference Example 10.

### Reference Example 11

The test sample of Example 5 (hyaluronic acid: 0.10% by mass; MgCl₂·6H₂O: 0.34% by mass) was used as the test sample of Reference Example 11.

### (Results)

According to the results of Reference Examples 8 and 9, in the case of the preparation using 0.50% by mass hyaluronic acid and the sodium salt, the compacted hyaluronic acid had an average particle diameter of 68.9 nm. On the other hand, in the case of the preparation using 0.50% by mass hyaluronic acid and the magnesium salt, the compacted hyaluronic acid had an average particle diameter of 295.9 nm.

Reference Example 9 has the same composition as Example 2 in Table 2. Conventionally, it has been said that the penetrability of hyaluronic acid into the skin decreases when the average particle diameter of hyaluronic acid exceeds 200 nm. However, from the results of Reference Example 9 and Example 2, it was found that a hyaluronic acid prepared using a predetermined amount of magnesium salt can be favorably allowed to penetrate into the skin even in cases where its average particle diameter is more than 200 nm.

The result of Reference Example 10 showed that, without the addition of the magnesium salt, the compaction of the hyaluronic acid did not occur, resulting in a bulky product with a size of 1,711 nm. It was found that further compaction of hyaluronic acid, to about 65.0 nm, can be achieved as in Reference Example 11 by including a magnesium salt and reducing the content of hyaluronic acid compared to Reference Example 9.

1. A cosmetic comprising:
a hyaluronic acid; and
a magnesium salt,
wherein the magnesium salt is contained at a concentration corresponding to an ionic strength of 0.01 or more, and
wherein the mass ratio of the magnesium salt to the hyaluronic acid is 1.0 or more.

2. The cosmetic according to claim 1, wherein the hyaluronic acid is contained at 0.005% by mass or more with respect to the total amount of the cosmetic.

3. The cosmetic according to claim 1, wherein the magnesium salt is magnesium chloride.

4. The cosmetic according to claim 1, wherein the weight average molecular weight of the hyaluronic acid is 10,000,000 or less.

5. The cosmetic according to claim 1, which is a leave-on type cosmetic.

6. The cosmetic according to claim 1, having an L value of 90 or more.

7. The cosmetic according to claim 1, for use in penetration of hyaluronic acid into body surface or body hair.

8. A beauty method comprising applying the cosmetic according to claim 1 to body surface or body hair.

9. The beauty method according to claim 8, wherein the application of the cosmetic to the body surface or body hair is carried out for two or more days.

10. The beauty method according to claim 8, wherein the application of the cosmetic to the body surface or body hair is carried out continuously for two or more days.
